(19) United States
(12) Patent Application Publication
(10) Pub. No.: US 2008/0149102 A1
(43) Pub. Date: Jun. 26, 2008

(54) Apparatus, method, and medium for leading respiration

(75) Inventors: Jong Pal Kim, Seoul (KR); Kun Soo Shin, Seongnam-si (KR); Soo Hyun Bae, Seoul (KR); Mi Hee Lee, Yongin-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/889,628

(22) Filed: Aug. 15, 2007

(30) Foreign Application Priority Data
Dec. 26, 2006 (KR) 10-2006-0134047

(51) Int. Cl. A61M 16/00

(52) U.S. Cl. 128/204.23

(57) ABSTRACT

A respiration leading apparatus, method, and medium, which can displace a respiration leading apparatus corresponding to a drive signal which corresponds to a selection input from a user, or displace a respiration leading apparatus generating the drive signal which corresponds to a biological signal of the user, thereby leading a respiration of the user, is provided. A respiration leading apparatus includes: a member; a selection module which receives a selection input from a user; a drive control module which outputs a drive signal corresponding to the selection input; and a drive module which displaces the member corresponding to the drive signal in order to lead a respiration of the user. A respiration leading apparatus, which can provide a user with structural displacement by a drive signal corresponding to biological information of the user, and be portable for the user, is provided.

### CROSS-REFERENCE TO RELATED APPLICATIONS

This application claims the priority benefit of Korean Patent Application No. 10-2006-0134047, filed on Dec. 26, 2006, in the Korean Intellectual Property Office, the disclosure of which is incorporated herein by reference.

### BACKGROUND

1. Field

Embodiments relate to a respiration leading apparatus, method, and medium, and more particularly, to a respiration leading apparatus, method, and medium, which can displace a respiration leading apparatus corresponding to a drive signal which corresponds to a selection input from a user, or displace a respiration leading apparatus by generating the drive signal which corresponds to a biological signal of the user, thereby leading a respiration of the user.

2. Description of the Related Art

Generally, apparatuses, which lead users into stable states and provide stimulation, are manually controlled, or provide the users with specific operations or stimulation based on previously programmed contents. However, there is a problem in that a leading apparatus of the former is manually controlled, and control operation is complex. Also, there is a problem that the apparatus is so large that the user cannot carry the apparatus. Also, since biological information corresponding to a biological parameter of the user is not reflected in a leading apparatus of the latter, the operation or the stimulation may provide the user with discomfort.

Therefore, a respiration leading apparatus and method, which can displace a respiration leading apparatus corresponding to a drive signal which corresponds to a selection input from a user, or displace a respiration leading apparatus by generating the drive signal which corresponds to a biological signal of the user, thereby leading a respiration of the user, is required.

### SUMMARY

An aspect of embodiments provides a respiration leading apparatus and method which can provide a user with structural displacement by a drive signal corresponding to biological information of the user, and be portable for the user.

An aspect of embodiments also provides a respiration leading apparatus in which progress of other operations cannot be obstructed while a respiration leading apparatus is used.

According to an aspect of embodiments, there is provided a respiration leading apparatus including: a member; a selection module which receives a selection input from a user; a drive control module which outputs a drive signal corresponding to the selection input; and a drive module which displaces the member corresponding to the drive signal in order to lead a respiration of the user.

According to another aspect of embodiments, there is provided a respiration leading apparatus including: a member; a detection module which measures a first biological signal of a user; a biological information extraction module which extracts first biological information from the first biological signal; a drive control module which outputs a drive signal corresponding to the first biological information; a drive module which displaces the member corresponding to the drive signal in order to lead a respiration of the user; and a relaxation determination module which compares the first biological information and second biological information corresponding to a second biological signal of the user measured in the detection module after a predetermined cyclic period, and controls the drive control module.

According to another aspect of the present invention embodiments, there is provided a respiration leading method by a respiration leading apparatus including a member, wherein the method includes: receiving a selection input from a user; generating a drive signal corresponding to the selection input, and displacing a member corresponding to the drive signal, and the drive signal has a predetermined period and a predetermined amplitude, and controls the member to displace into either an inhalation pattern or an exhalation pattern.

According to another aspect of the present invention embodiments, there is provided a respiration leading method by a respiration leading apparatus including a member, the method including receiving a first biological signal from a user; extracting first biological information from the first biological signal; outputting a drive signal corresponding to the first biological information; displacing the member corresponding to the drive signal in order to lead a respiration of the user; and comparing the first biological information and second biological information corresponding to a second biological signal of the user measured in a respiration leading apparatus after a predetermined cycling period, outputting a updated drive signal, and displacing the member corresponding to the updated drive signal.

According to another aspect of the present invention embodiments, there is provided a respiration leading apparatus including a member; a selection module to receive a selection input from a user; a detection module to measure a first biological signal of a user; a biological information extraction module to extract first biological information from the first biological signal; a drive control module to output a drive signal corresponding to one of the selection input from the user or the first biological information; and a drive module to displace the member corresponding to the drive signal in order to lead a respiration of the user.

According to another aspect of the present invention embodiments, there is provided a respiration leading method by a respiration leading apparatus including a member, wherein the method includes receiving one of a selection input from a user or a first biological signal from a user; extracting first biological information from the first biological signal if the first biological signal is received; outputting a drive signal corresponding to the selection input or first biological information; and displacing a member according to the drive signal.

According to another aspect of embodiments, there is provided at least one computer readable medium storing computer readable instructions to implement methods of embodiments.

### BRIEF DESCRIPTION OF THE DRAWINGS

The above and/or other aspects, features, and advantages of exemplary embodiments will become apparent and more readily appreciated from the following detailed description, taken in conjunction with the accompanying drawings of which:

FIG. 1 is a block diagram illustrating components of a respiration leading apparatus according to an exemplary embodiment;

FIG. 2 illustrates a drive signal which is outputted by a drive control module and corresponds to a selection input of a user according to an exemplary embodiment;

FIG. 3 is a block diagram illustrating components of a respiration leading apparatus according to another exemplary embodiment;

FIG. 4 is a flowchart illustrating a respiration leading method by a respiration leading apparatus including a member according to an exemplary embodiment;

FIG. 5 is a flowchart illustrating a respiration leading method by a respiration leading apparatus including a member according to another exemplary embodiment;

FIG. 6 is a diagram illustrating a drive control module including an adaptive filter which extracts a respiration signal of a user from a drive signal according to an exemplary embodiment;

FIG. 7 is a flowchart illustrating a respiration leading method by a respiration leading apparatus of a specific example according to an exemplary embodiment; and

FIG. 8 is a diagram illustrating a form of a displacement of a respiration leading apparatus included in a form of a doll, according to an exemplary embodiment.

### DETAILED DESCRIPTION OF EMBODIMENTS

Reference will now be made in detail to exemplary embodiments, examples of which are illustrated in the accompanying drawings, wherein like reference numerals refer to the like elements throughout. Exemplary embodiments are described below by referring to the figures.

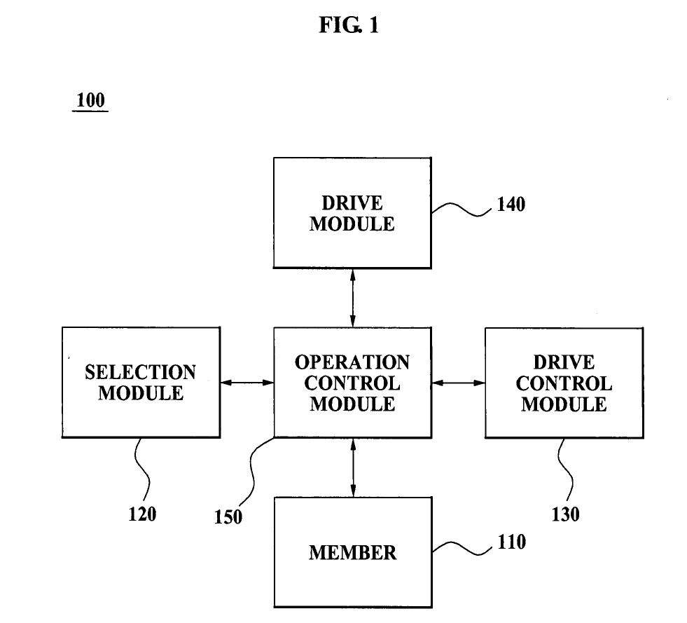

FIG. 1 is a block diagram illustrating components of a respiration leading apparatus 100 according to an exemplary embodiment.

Referring to FIG. 1, the respiration leading apparatus 100 according to the present exemplary embodiment includes a member 110, a selection module 120, a drive control module 130, and a drive module 140. The respiration leading apparatus 100 may induce inhalation and may induce exhalation.

The member 110 may correspond to a portion including an external form of the respiration leading apparatus 100 and a framework described above, and a material configuring an external form of an apparatus for leading to an existing stable state may be used for a material included in the member 110.

The selection module 120 receives a selection input from a user, and the selection input includes a configuration that enables the user of the respiration leading apparatus 100 to select. The selection module 120 may be expressed in a predetermined display device in order to enable easy recognition by the user, and enables the user to select a program type via the display device. Also, the selection module 120 may include a button in which a program providing one-sided stimulation without a display device is recorded.

The drive control module 130 outputs a drive signal corresponding to the selection input that the selection module 120 receives. The drive signal has a predetermined period and a predetermined amplitude, and controls the member 110 to displace into either an inhalation pattern or an exhalation pattern. The drive control module 130 receives the selection input, which is inputted and selected by a user and received via the selection module 120, outputs a drive signal corresponding to the selection input, and controls displacement of the drive module 140.

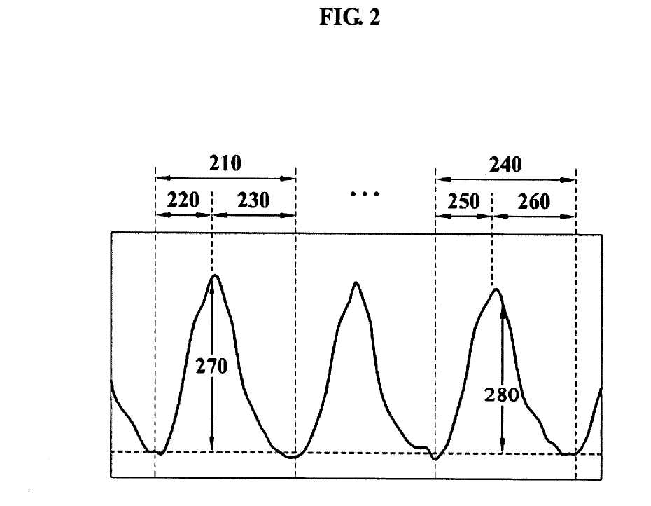

The drive module 140 displaces the member 110 corresponding to the drive signal, which is outputted by the drive control module 130, in order to lead a respiration of the user. An apparatus generating predetermined structural displacement such as either an air pump or a motor driving apparatus may be unrestrictedly used for the drive module 140. The drive signal is described with reference to FIG. 2.

Also, a respiration leading apparatus 100 according to the present exemplary embodiment may further include an operation control module 150 controlling the member 110, the selection module 120, the drive control module 130, and the drive module 140 which are described above.

The user may conveniently carry the respiration leading apparatus 100, and perform a selection input by the selection module 120, and be led to a stable state by structural displacement of the drive module 140 via the member 110 by a drive signal corresponding to the selection input, in the respiration leading apparatus 100 according to the present exemplary embodiment. The respiration leading apparatus 100 formed by the member 110 may have various forms depending upon a taste of the user such as a doll, a bed mat, and a pillow.

FIG. 2 illustrates a drive signal which is outputted by a drive control module and corresponds to a selection input of a user according to an exemplary embodiment.

Referring to FIG. 2, the drive signal according to the present exemplary embodiment is outputted by the drive control module 130 corresponding to the selection input received by the selection module 120, and the drive module 140 displaces the member 110 corresponding to the drive signal. The user performs a selection input via the selection module 120 in order to obtain an output that the drive signal desires. Items in which the user may perform a selection input via the selection module 120 may be a period of time of one initial respiration 210, an inhalation period of time of initial respiration 220, an exhalation period of time of initial respiration 230, a period of time of one n-th respiration 240, an inhalation period of time of n-th respiration 250, an exhalation period of time of n-th respiration 260, a leading amplitude of initial respiration 270, a leading amplitude of n-th respiration 280, and the like. Specifically, the user is not unilaterally provided with a program, similar to a leading apparatus according to a related art, however, the user may be provided with structural displacement appropriate for the user's state according to the user's judgment, and be smoothly led to a stable state.

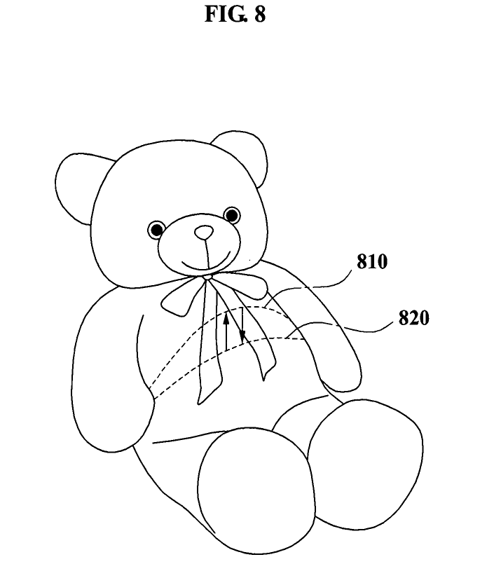

Displacement which may be generated by the drive signal described above in the respiration leading apparatus 100 according to the present exemplary embodiment is described with reference to FIG. 8.

FIG. 8 is a diagram illustrating a form of a displacement of a respiration leading apparatus included in a form of a doll according to an exemplary embodiment.

Referring to FIGS. 1, 2 and 8, a respiration leading apparatus 100 according to the present exemplary embodiment may include an external appearance of a doll form by a member 110, and the member 110 generates displacement corresponding to a drive signal corresponding to a selection input of a user by a drive module 140. As illustrated in FIG. 2, the user may perform, via the selection module 120, a selection input corresponding to a period of time of one initial respiration 210, an inhalation period of time of initial respiration 220, an exhalation period of time of initial respiration 230, a period of time of one n-th respiration 240, an inhalation period of time of n-th respiration 250, an exhalation period of time of n-th respiration 260, a leading amplitude of initial respiration 270, a leading amplitude of n-th respiration 280, and the like. Also, items selectively inputted are reflected in a drive signal, and the drive module 140 generates displacement corresponding to the drive signal. Specifically, the drive signal has a predetermined period and a predetermined amplitude illustrated in FIG. 2, and controls the member 110 to displace into either an inhalation pattern or an exhalation pattern. For example, the respiration leading apparatus, e.g. a doll may copy a respiration of inhalation 810, and have an inflated belly in the case of inhalation, and may copy a respiration of exhalation 820, and have a shrunken belly in the case of exhalation. Heights of amplitudes 810 and 820 in which the inhalation and the exhalation are respired, a period, and the like are selected by the user, as illustrated in FIG. 2, and is controlled by the drive signal in which the inputted selection input is reflected.

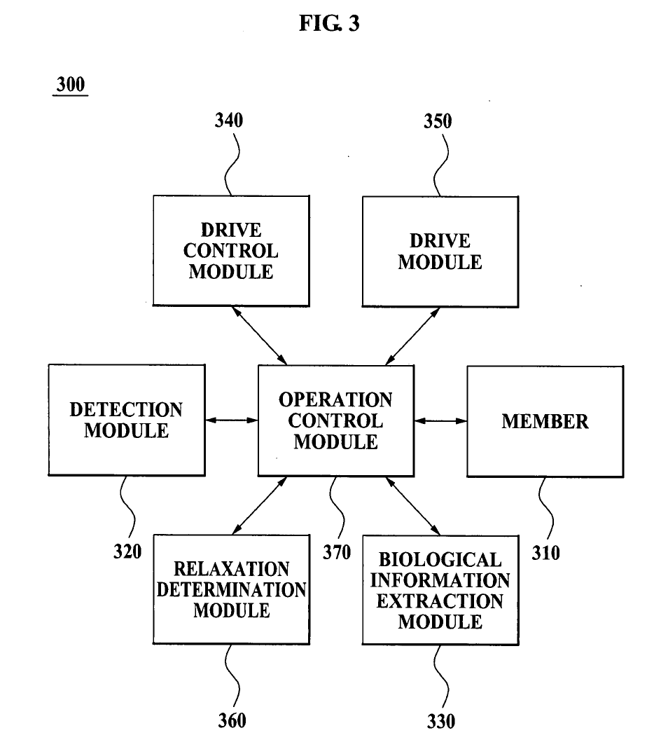

FIG. 3 is a block diagram illustrating components of a respiration leading apparatus according to another exemplary embodiment.

Referring to FIG. 3, a respiration leading apparatus 300 according to the present exemplary embodiment includes a member 310, a detection module 320, a biological information extraction module 330, a drive control module 340, a drive module 350, and a relaxation determination module 360.

The member 310 may correspond to a portion including an external form of the respiration leading apparatus 300 and a framework, and a material configuring an external form of an apparatus for leading a user to an existing stable state may be used for a material included in the member 310.

The detection module 320 measures a first biological signal of a user. The first biological signal may be any one from among an electroencephalogram signal, an electrocardiogram signal, an electromyogram signal, a heart rate signal, a body temperature, and skin resistance. Also, the detection module 320 may be any one module from among a heart rate measurement module, a skin electrical resistance measurement module, and an electromyogram measurement module. The detection module 320 measures a biological signal such as an electroencephalogram signal, an electrocardiogram signal, an electromyogram signal, a heart rate signal, a body temperature, and skin resistance via an apparatus such as a heart rate measurement module, a skin electrical resistance measurement module, and an electromyogram measurement module.

The biological information extraction module 330 extracts first biological information from the first biological signal. The first biological information includes information with respect to a drive signal to be outputted from the drive control module 340 of the respiration leading apparatus 300 according to the present exemplary embodiment. The biological information extraction module 330 extracts first biological information, which corresponds to basic information required for generating and outputting a drive signal, from the first biological signal such as an electroencephalogram signal, an electrocardiogram signal, an electromyogram signal, a heart rate signal, a body temperature, and skin resistance measured by the detection module 320.

The drive control module 340 outputs a drive signal corresponding to the first biological information. Specifically, a drive signal appropriate for the first biological information individualized for the user may be outputted, and displacement of the member 310 may be controlled by a user-oriented drive signal. The drive signal has a predetermined period and a predetermined amplitude, and controls the member 310 to displace into either an inhalation pattern or an exhalation pattern, as illustrated in FIG. 2.

The drive module 350 displaces the member 310 corresponding to the drive signal, which is outputted by the drive control module 130, in order to lead a respiration of the user. The drive signal has a predetermined period and a predetermined amplitude, and controls the member 310 to displace into either an inhalation pattern or an exhalation pattern. An apparatus generating predetermined structural displacement such as with either an air pump or a motor driving apparatus may be freely used for the drive module 350, as illustrated in FIG. 1.

The relaxation determination module 360 compares the first biological information and second biological information corresponding to a second biological signal of the user measured in the detection module 320 after a predetermined cyclic period, and controls the drive control module 340. Specifically, the relaxation determination module 360 controls a cyclic period depending upon a stable state of the user, and whether a relaxation degree is improved, by comparing the first biological information and the second biological information. Specifically, when the relaxation degree is improved, increasing and decreasing periods 210 and 240 of a led respiration illustrated in FIG. 2, i.e. a cyclic period, may be repeated. When the relaxation degree is not improved, the relaxation determination module 360 determines that the user feels a burden during a led respiration period, and the relaxation determination module 360 may decrease the led respiration period. Also, when the relaxation degree is at a maximum, the led respiration period may be maintained.

The relaxation determination module 360 determines a relaxation level using a first relaxation index calculated from the first biological information, and a second relaxation index calculated from the second biological information, and supplies a feedback signal corresponding to the relaxation level for the drive control module 340. The first relaxation index and the second relaxation index are calculated from the first biological information and the second biological information, and may be expressed in an index illustrating a relaxation degree. The relaxation determination module 360 may determine the relaxation level according to a predetermined feedback period, and the feedback period may be controlled according to the determined relaxation level. The relaxation level may correspond to at least two discrete values calculated based on at least one threshold value.

Also, the drive signal has a predetermined period and a predetermined amplitude, and controls the member 310 to displace into either an inhalation pattern or an exhalation pattern. The drive signal may change the amplitude corresponding to the relaxation level of the user. Also, the relaxation determination module 360 may determine the relaxation level according to a predetermined feedback period, and the feedback period may be controlled according to the determined relaxation level.

As a specific example, when the first biological signal or the second biological signal is a heart rate signal, the first biological information or the second biological information may be a heart rate. When the user is relaxed, a heart rate corresponding to biological information is generally low, and the first relaxation index or the second relaxation index may be defined as a value subtracting a heart rate corresponding to the first biological information or the second biological information from a determination standard value, e.g. 200 (relaxation index=200−biological information (heart rate)). When a heart rate from the first biological information and the second biological information is increased, the first relaxation index or the second relaxation index is decreased. Conversely, when a heart rate is decreased, the relaxation index is increased. Specifically, the first relaxation index or the second relaxation index may be an index expressing a degree of a state in which the user is stabilized. The relaxation level of the user is determined using the first relaxation index and the second relaxation index, and the respiration leading apparatus provides displacement depending upon the relaxation level. The relaxation level may correspond to at least two discrete values calculated based on at least one threshold value, and has a predetermined period and a predetermined amplitude. Also, an amplitude of displacement may be controlled corresponding to the relaxation level of the user by the drive signal controlling the member to displace into either an inhalation pattern or an exhalation pattern.

As another example, a heart rate variability (HRV) may be used for the relaxation index. When an increase and decrease degree of the HRV is great, i.e. when a difference between the first relaxation index and the second relaxation index is great, the relaxation level of the user may be measured as high. The first biological signal or the second biological signal described above may include an electroencephalogram signal, an electrocardiogram signal, an electromyogram signal, a body temperature, skin resistance, and the like, in addition to a heart rate signal corresponding to various users. The relaxation index may be variously expressed corresponding to the various biological signals.

According to exemplary embodiments, there is provided a respiration leading apparatus, which can provide a user with structural displacement appropriate for the user by a drive signal corresponding to biological information of the user, and be portable for the user.

Also, the respiration leading apparatus 300 according to the present exemplary embodiment may further include an operation control module 370 controlling the member 310, the detection module 320, the biological information extraction module 330, the drive control module 340, the drive module 350, and the relaxation determination module 360.

Also, a respiration leading apparatus 300 according to the present exemplary embodiment may further include a display module (not illustrated) which displays the first biological information or the second biological information of the user. The user may check the user's own degree of stabilization, and may check the user's own degree of stabilization after a predetermined cycling period of the respiration leading apparatus 300 by the display module.

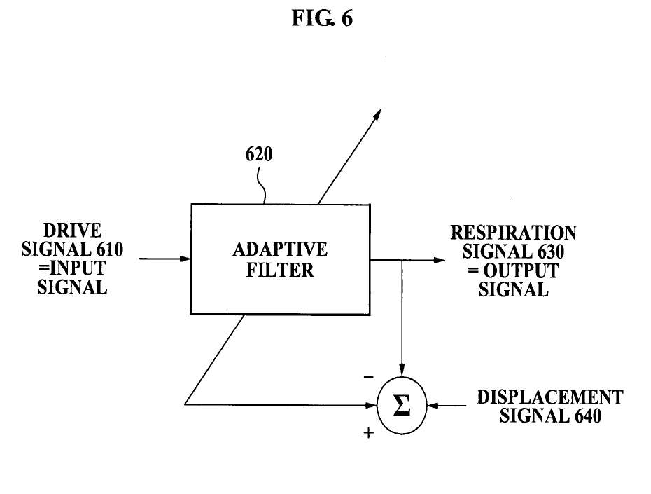

The drive control module 340 may further include an adaptive filter which filters and outputs the drive signal. The adaptive filter is described with reference to FIG. 6.

FIG. 6 is a diagram illustrating a drive control module including an adaptive filter 620 which extracts a respiration signal of a user from a drive signal according to an exemplary embodiment.

Generally, the adaptive filter is a filter providing an output signal (a respiration signal 630) corresponding to a desirable signal using an adaptive learning method during a process in which an input signal (a drive signal 610) is received, and processes the input signal into a desirable signal. The adaptive filter is generally used for a case that a characteristic of the filter is constantly changed to adapt to a signal and other states, or a case that frequency bands of a signal and noises are overlapped, or a frequency characteristic of noises is unknown, or the characteristic is changed over time.

The detection module 320 may further include a dislocation sensing module (not illustrated) which measures a dislocation signal corresponding to dislocation of the member 310, and the drive control module 340 further includes an adaptive filter which filters the drive signal and the dislocation signal. Specifically, a biological signal of a user is measured providing a sensor or a measurement belt with a user's specific region according to a related art, however, a drive signal 610 and a displacement signal 640 measured from the user via the dislocation sensing module of the detection module 320 are adaptively filtered, and a respiration signal 630 of the user is measured, and a feedback to the described process is performed according to the present exemplary embodiment, thereby effectively leading a respiration appropriate for the user.

The adaptive filter 620 according to the present exemplary embodiment may be a combination-type adaptive filter using either a Least Mean Square (LMS) algorithm or a Recursive Least Square (RLS) algorithm.

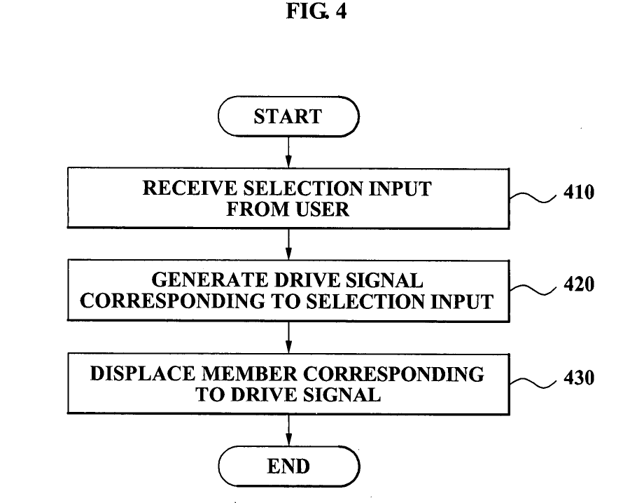

FIG. 4 is a flowchart illustrating a respiration leading method by a respiration leading apparatus including a member according to an exemplary embodiment.

Referring to FIG. 4, a selection input is first received from a user in operation 410, and a drive signal is generated corresponding to the selection input in operation 420, and a member is displaced corresponding to the drive signal in operation 430. The drive signal has a predetermined period and a predetermined amplitude, and controls the member to displace into either an inhalation pattern or an exhalation pattern, similar to the description above. A displacement degree of the drive signal and the member is described in FIGS. 2 and 3.

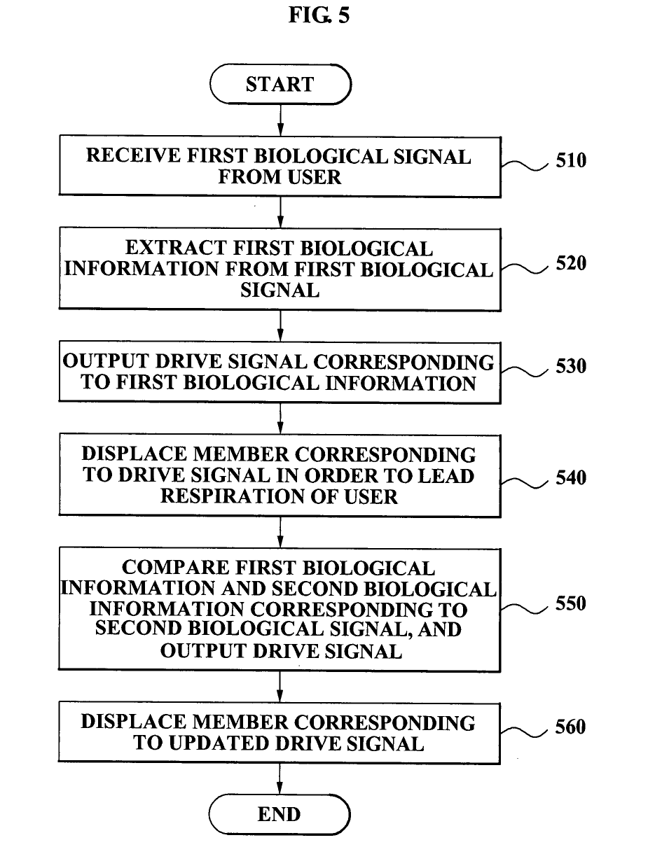

FIG. 5 is a flowchart illustrating a respiration leading method by a respiration leading apparatus including a member according to another exemplary embodiment.

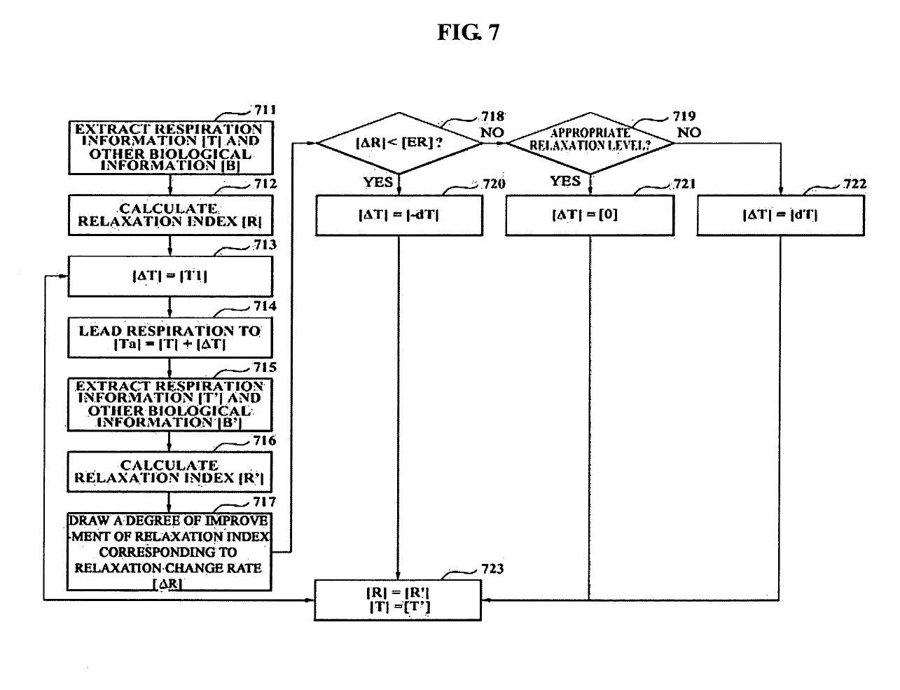

Referring to FIG. 5, a first biological signal is first received from a user in operation 510. The first biological signal may be any one from among an electroencephalogram signal, an electrocardiogram signal, an electromyogram signal, a heart rate signal, a body temperature, and skin resistance, similar to the description above. Next, first biological information is extracted from the first biological signal in operation 520. The first biological information may include information with respect to a drive signal to be outputted from the respiration leading apparatus. Specifically, the first biological information may correspond to basic information required for generating and outputting a drive signal, from the first biological signal such as an electroencephalogram signal, an electrocardiogram signal, an electromyogram signal, a heart rate signal, a body temperature, and skin resistance. Next, a drive signal corresponding to the first biological information is outputted in operation 530. The drive signal has a predetermined period and a predetermined amplitude, and controls the member to displace into either an inhalation pattern or an exhalation pattern, similar to the description above. Next, the member is displaced corresponding to the drive signal in order to lead a respiration of the user in operation 540, and the first biological information and second biological information corresponding to a second biological signal of the user measured in a respiration leading apparatus after a predetermined cycling period are compared, and an updated drive signal is outputted in operation 550. The member corresponding to the updated drive signal is displaced in operation 560. A detailed respiration leading method is described with reference to an example in FIG. 7.

FIG. 7 is a flowchart illustrating a respiration leading method by a respiration leading apparatus of a specific example according to an exemplary embodiment.

Referring to FIG. 7, respiration information [T] with respect to a user, and other biological information [B] are extracted from a detection module of the respiration leading apparatus and a biological information extraction module in operation 711. Here, [ ] indicates a vector amount. Next, a relaxation index [R] is calculated from respiration information [T] or other biological information [B] operation 712. It is obvious to those of ordinary skill in the art that the relaxation index [R] includes information such as a heart rate (HR), HRV, and the like, similar to the description above. Next, a predetermined numerical value [T1] is determined as a predetermined addition period [ΔT] in operation 713, and a cycling period [Ta] is established by adding an addition period [ΔT] and respiration information [T], and a respiration is led by a drive module in operation 714. Next, respiration information [T′] and other biological information [B′], measured from the detection module after a cycling period [Ta], are extracted in operation 715, and relaxation index [R′] is calculated from respiration information [T] or other biological information [B], and respiration information [T′] or other biological information [B′] in operation 716. A degree of improvement of a relaxation index [ΔR] is drawn in operation 717. The improvement degree of the relaxation index may correspond to a relaxation change rate in the present example. Next, the relaxation change rate [ΔR] is compared with a predetermined determination value [ER] in operation 718, and when the relaxation change rate [ΔR] is less than the determination value [ER], respiration leading is weakened, the addition period [ΔT] is reduced to [dT], thereby reducing the burden of the user due to respiration leading, and continuing processing to operation 723. However, when the relaxation change rate [ΔR] is greater than or equal to the determination value [ER], whether an appropriate relaxation level is reached is determined in operation 719, and when an appropriate relaxation level is reached, an addition period [ΔT] is controlled to [0] in order to maintain a current respiration leading in operation 721, and continues processing to operation 723. However, when it is determined that an appropriate relaxation level is not reached in operation 719, an addition period [ΔT] is increased to [dT], and respiration leading is strengthened, and continues processing to operation 723 for additional respiration leading in operation 722. Next, in operation 723, respiration information [T′] of the user, or other biological information [B′] is replaced with the first drawn respiration information [T] or the first drawn other biological information [B], and continues processing to operation 713. Also, a feedback is constantly performed, and the above process is performed until an appropriate relaxation level is reached. According to a respiration leading method by a respiration leading apparatus illustrated in FIG. 7, the user is provided with displacement of respiration leading specialized for the user and continuously adapts the respiration leading until an appropriate relaxation level is reached.

In addition to the above-described exemplary embodiments, exemplary embodiments can also be implemented by executing computer readable code/instructions in/on a medium/media, e.g., a computer readable medium/media. The medium/media can correspond to any medium/media permitting the storing and/or transmission of the computer readable code/instructions. The medium/media may also include, alone or in combination with the computer readable code/instructions, data files, data structures, and the like. Examples of code/instructions include both machine code, such as produced by a compiler, and files containing higher level code that may be executed by a computing device and the like using an interpreter. In addition, code/instructions may include functional programs and code segments.

The computer readable code/instructions can be recorded/transferred in/on a medium/media in a variety of ways, with examples of the medium/media including magnetic storage media (e.g., floppy disks, hard disks, magnetic tapes, etc.), optical media (e.g., CD-ROMs, DVDs, etc.), magneto-optical media (e.g., floptical disks), hardware storage devices (e.g., read only memory media., random access memory media, flash memories, etc.) and storage/transmission media such as carrier waves transmitting signals, which may include computer readable code/instructions, data files, data structures, etc. Examples of storage/transmission media may include wired and/or wireless transmission media. For example, storage/transmission media may include optical wires/lines, waveguides, and metallic wires/lines, etc. including a carrier wave transmitting signals specifying instructions, data structures, data files, etc. The medium/media may also be a distributed network, so that the computer readable code/instructions are stored/transferred and executed in a distributed fashion. The medium/media may also be the Internet. The computer readable code/instructions may be executed by one or more processors. The computer readable code/instructions may also be executed and/or embodied in at least one application specific integrated circuit (ASIC) or Field Programmable Gate Array (FPGA).

In addition, one or more software modules or one or more hardware modules may be configured in order to perform the operations of the above-described exemplary embodiments.

The term “module”, as used herein, denotes, but is not limited to, a software component, a hardware component, a plurality of software components, a plurality of hardware components, a combination of a software component and a hardware component, a combination of a plurality of software components and a hardware component, a combination of a software component and a plurality of hardware components, or a combination of a plurality of software components and a plurality of hardware components, which performs certain tasks. A module may advantageously be configured to reside on the addressable storage medium/media and configured to execute on one or more processors. Thus, a module may include, by way of example, components, such as software components, application specific software components, object-oriented software components, class components and task components, processes, functions, operations, execution threads, attributes, procedures, subroutines, segments of program code, drivers, firmware, microcode, circuitry, data, databases, data structures, tables, arrays, and variables. The functionality provided for in the components or modules may be combined into fewer components or modules or may be further separated into additional components or modules. Further, the components or modules can operate at least one processor (e.g. central processing unit (CPU)) provided in a device. In addition, examples of a hardware components include an application specific integrated circuit (ASIC) and Field Programmable Gate Array (FPGA). As indicated above, a module can also denote a combination of a software component(s) and a hardware component(s). These hardware components may also be one or more processors.

The computer readable code/instructions and computer readable medium/media may be those specially designed and constructed for the purposes of exemplary embodiments, or they may be of the kind well-known and available to those skilled in the art of computer hardware and/or computer software.

According to exemplary embodiments, there is provided a respiration leading apparatus, method, and medium, which can provide a user with structural displacement by a drive signal corresponding to biological information of the user, and be portable for the user.

Also, according to exemplary embodiments, there is provided a respiration leading apparatus, in which progress of other operations cannot be obstructed while a respiration leading apparatus is used.

Although a few exemplary embodiments have been shown and described, it would be appreciated by those skilled in the art that changes may be made to these exemplary embodiments, the scope of which is defined by the claims and their equivalents.

What is claimed is:

1. A respiration leading apparatus comprising:
a member;
a selection module which receives a selection input from a user;
a drive control module which outputs a drive signal corresponding to the selection input; and
a drive module which displaces the member corresponding to the drive signal in order to lead a respiration of the user.

2. The respiration leading apparatus of claim 1, wherein the drive signal has a predetermined period and a predetermined amplitude, and controls the member to displace into either an inhalation pattern or an exhalation pattern.

3. The respiration leading apparatus of claim 1, wherein the drive module is either an air pump or a motor driving apparatus.

4. A respiration leading apparatus comprising:
a member;
a detection module which measures a first biological signal of a user;
a biological information extraction module which extracts first biological information from the first biological signal;
a drive control module which outputs a drive signal corresponding to the first biological information;
a drive module which displaces the member corresponding to the drive signal in order to lead a respiration of the user; and
a relaxation determination module which compares the first biological information and second biological information corresponding to a second biological signal of the user measured in the detection module after a predetermined cyclic period, and controls the drive control module based on the comparison.

5. The respiration leading apparatus of claim 4, wherein the relaxation determination module determines a relaxation level using a first relaxation index calculated from the first biological information, and a second relaxation index calculated from the second biological information, and supplies a feedback signal corresponding to the relaxation level for the drive control module.

6. The respiration leading apparatus of claim 5, wherein the relaxation determination module determines the relaxation level according to a predetermined feedback period, and the feedback period is controlled according to the determined relaxation level.

7. The respiration leading apparatus of claim 4, wherein the drive signal has a predetermined period and a predetermined amplitude, and controls the member to displace into either an inhalation pattern or an exhalation pattern.

8. The respiration leading apparatus of claim 7 further comprising:
a display module which displays the first biological information or the second biological information of the user.

9. The respiration leading apparatus of claim 4, wherein the drive module is either an air pump or a motor driving apparatus.

10. The respiration leading apparatus of claim 4, wherein the detection module is any one module from among a heart rate measurement module, a skin electrical resistance measurement module, and an electromyogram measurement module.

11. The respiration leading apparatus of claim 4, wherein the first biological signal or the second biological signal is any one from among an electroencephalogram signal, an electrocardiogram signal, an electromyogram signal, a heart rate signal, a body temperature, and skin resistance.

12. The respiration leading apparatus of claim 4, wherein the detection module further comprises a dislocation sensing module which measures a dislocation signal corresponding to a dislocation of the member,
and the drive control module further comprises an adaptive filter which filters the drive signal and the dislocation signal.

13. The respiration leading apparatus of claim 12, wherein the adaptive filter is a combination-type adaptive filter using either a Least Mean Square (LMS) algorithm or a Recursive Least Square (RLS) algorithm.

14. The respiration leading apparatus of claim 5, wherein the relaxation level corresponds to at least two discrete values calculated based on at least one threshold value.

15. The respiration leading apparatus of claim 5, wherein the drive signal has a predetermined period and a predetermined amplitude, and controls the member to displace into either an inhalation pattern or an exhalation pattern, and
the drive signal changes the amplitude corresponding to the relaxation level of the user.

16. A respiration leading method by a respiration leading apparatus including a member, wherein the method comprises:
receiving a selection input from a user;
generating a drive signal corresponding to the selection input, and displacing a member corresponding to the drive signal,
and the drive signal has a predetermined period and a predetermined amplitude, and controls the member to displace into either an inhalation pattern or an exhalation pattern.

17. A respiration leading method by a respiration leading apparatus including a member, the method comprising:
receiving a first biological signal from a user;
extracting first biological information from the first biological signal;
outputting a drive signal corresponding to the first biological information;
displacing the member corresponding to the drive signal in order to lead a respiration of the user; and
comparing the first biological information and second biological information corresponding to a second biological signal of the user measured in a respiration leading apparatus after a predetermined cycling period, outputting a updated drive signal, and displacing the member corresponding to the updated drive signal.

18. At least one computer readable medium storing computer readable instructions that control at least one processor to implement the method according to claim 16.

19. At least one computer readable medium storing computer readable instructions that control at least one processor to implement the method of claim 17.

20. A respiration leading apparatus comprising:
a member;
a selection module to receive a selection input from a user;
a detection module to measure a first biological signal of a user;
a biological information extraction module to extract first biological information from the first biological signal;
a drive control module to output a drive signal corresponding to one of the selection input from the user or the first biological information; and
a drive module to displace the member corresponding to the drive signal in order to lead a respiration of the user.

21. The respiration leading control apparatus of claim 20, further comprising a relaxation determination module to compare the first biological information and second biological information corresponding to a second biological signal of the user measured in the detection module after a predetermined cyclic period, and controls the drive control module.

22. A respiration leading method by a respiration leading apparatus including a member, wherein the method comprises:
receiving one of a selection input from a user or a first biological signal from a user;
extracting first biological information from the first biological signal if the first biological signal is received;
outputting a drive signal corresponding to the selection input or first biological information; and
displacing a member according to the drive signal.

23. The respiration leading control method of claim 22, further comprising comparing the first biological information and second biological information corresponding to a second biological signal of the user measured in a respiration leading apparatus after a predetermined cycling period, outputting a updated drive signal, and displacing the member corresponding to the updated drive signal.

24. At least one computer readable medium storing computer readable instructions that control at least one processor to implement the method of claim 22.
